(19) United States
(12) Patent Application Publication
(10) Pub. No.: US 2005/0154239 A1
(43) Pub. Date: Jul. 14, 2005

(54) Methods for preparing 1,3 butylene glycol

(76) Inventors: Kenneth Allen Windhorst, Portland, TX (US); Richard D. Guajardo, Kingsville, TX (US)

Correspondence Address: M. Susan SPIERING;c/o Celanese Ltd. / IP Legal Dept., IZIP 701, P.O. Box 428, Bishop, TX, 78343, US

(21) Appl. No.: 10/753,745

(22) Filed: Jan. 8, 2004

(51) Int. Cl. C07C 29/14

(52) U.S. Cl. 568/863

(57) ABSTRACT

1,3 butylene glycol, prepared through an intermediate aldol condensation reaction of acetaldehyde, is produced at increased yield efficiencies. The efficiencies are achieved by utilizing an acetaldehyde having low carboxylic concentrations. The aldol condensation takes place in the presence of an alkali agent at a concentration of about 2 ppm to about 10 ppm to produce a 3-hydroxybutanal intermediate product that is hydrogenated in the presence of a Raney nickel catalyst to yield 1,3 butylene glycol at efficiency yields of greater than about 75%.

### FIELD OF THE DISCLOSURE

This disclosure relates to processes for preparing 1,3 butylene glycol.

### BACKGROUND INFORMATION

1,3-butylene glycol is a widely used industrial organic compound. It is viscous, colorless, transparent, and low odor, and is capable of producing chemically-stable derivatives. 1,3 butylene glycol is a useful compound as a solvent for coatings, starting materials for various synthetic resins and surfactants, a high-boiling-point solvent and antifreeze, food supplements, animal food supplements, a humectant for tobacco composition and an intermediate for preparation of various other compounds.

There are various processes recognized for producing 1,3 butylene glycol commercially. U.S. Pat. No. 6,376,725 discloses a process for producing 1,3 butylene glycol though a liquid phase hydrogenation of acetaldol (3-hydroxybutanal or aldol) in the presence of a Raney nickel catalyst. Acetaldol is commonly produced through the aldol condensation of two molecules of acetaldehyde.

U.S. Pat. Nos. 5,345,004 and 5,583,270 disclose process for producing 1,3 butylene glycol in three step processes including an aldol condensation of acetaldehyde to aldoxane, followed by decomposition of the aldoxane to obtain paraldol which is in turn hydrogenated to produce 1,3 butylene glycol.

Conventional industrial processes produce 1,3 butylene glycol at a yield efficiency of less than 75%.

As exemplified by the above-identified disclosures, most commercial processes for producing 1,3 butylene glycol make use of acetaldehyde as a compound for producing intermediate products used in the production of 1,3 butylene glycol. Acetaldehyde is a well known compound, useful in the production of other compounds such as acetic acid, acetic anhydride, n-butanol, 2-ethylhexanol, peracetic acid, pentaerythritol, pyridines, chloral, and trimethylolpropane. Acetaldehyde has been produced conventionally by methods such as the hydration of acetylene or the oxidation of ethylene, but such methods have their limitations, particularly as to cost and it would be desirable to find a more economic method for the preparation of this compound.

As disclosed in U.S. Pat. No. 4,525,481, many processes have been disclosed for reacting methanol and other C-1 derived chemicals such as formaldehyde and methyl acetate with carbon monoxide and hydrogen in the presence of catalyst systems to produce a wide variety of compounds.

U.S. Pat. No. 4,151,208 teaches that acetaldehyde may be selectively produced by contacting methanol, hydrogen and carbon monoxide with cobalt (II) meso-tetraaromatic porphine and an iodine promoter.

Other examples for acetaldehyde synthesis from methanol and CO/H₂ are seen in U.S. Pat. Nos. 4,239,704; 4,239,705; 4,225,517; 4,201,868; 4,337,365; 4,306,091 and 4,348,541, J. Molecular Catalysis, Vol. 17 (1982), 339-347, Organometallics, Vol. 2, No. 12 (1983), 1881, and European Patents 0 011 042, 0 022 735 and 0 037 588. Most of these processes use catalyst systems with a homogeneous cobalt and/or ruthenium compound with an iodine promoter.

A palladium catalyst with iodide promoter is disclosed U.S. Pat. No. 4,302,611 for acetaldehyde synthesis from the reaction of methyl acetate and CO/H.₂.

U.S. Pat. Nos. 4,291,179 and 4,267,384 disclose the conversion of formaldehyde into acetaldehyde by the use of rhodium and ruthenium catalysts.

A general disadvantage of all commercial processes for acetaldehyde production is that they produce a wide variety of by-products such as higher molecular weight alcohols, aldehydes, hydrocarbons, carboxylic acids, and esters. For example, acetic acid is a common impurity in acetaldehyde available for industrial processes, including production of 1,3 butylene glycol.

Typical specifications for acetic acid concentrations in acetaldehyde for industrial use range from 0.05 wt. % to 0.1 wt. %, based upon the total weight of the acetaldehyde product.

### BRIEF DESCRIPTION OF THE DISCCLOSURE

This disclosure relates to processes for preparing 1,3 butylene glycol through process steps including an aldol condensation of acetaldehyde and/or hydrogenation of 3-hydroxybutanal. It has been unexpectedly discovered that yield efficiencies for preparing 1,3 butylene glycol can be dramatically increased by aldol condensation of acetaldehyde having a carboxylic acid content of less than 0.04 wt. % based upon the weight of the acetaldehyde. The aldol condensation takes place in the presence of an alkali agent, acting as a catalyst, at a concentration of about 2 to about 10 ppm to produce a 3-hydroxybutanal (acetaldol) intermediate product that is hydrogenated in the presence of a Raney Nickel catalyst to yield 1,3 butylene glycol at efficiency yields of greater than about 75%. Other traditionally more expensive catalyst systems such as palladium, platinum, and ruthenium may also be used although economic may make it difficult to use these catalyst systems commercially.

It has been discovered that the presence of carboxylic acids in the acetaldehyde neutralize the alkali agent to form salts. The salts, in turn, appear to catalyze the formation of by-products. The improvement in yield efficiency is believed to result from the minimization of salt formation and correspondingly the formation of by-products to lower production yields.

### BRIEF DESCRIPTION OF THE DRAWINGS

FIG. 1 is a schematic diagram of an embodiment of the processes described herein.

FIG. 2 is a plot of the yield efficiencies and corresponding acetaldehyde acid concentrations for commercial production of 1,3 butylene glycol over a 113 day period.

### DETAILED DISCLOSURE

Typical commercial production yields for 1,3 butylene glycol processes are less than 75%. This disclosure relates to improved processes for preparing 1,3 butylene glycol at high yield efficiencies. The improved processes increase yield efficiencies by minimizing the production of by-products previously accepted as inevitable in the production of 1,3 butylene glycol.

More particularly, this disclosure relates to methods for preparing 1,3 butylene glycol through process steps including an aldol condensation of acetaldehyde. The aldol condensation of the acetaldehyde produces 3-hydroxybutanl (acetaldol or aldol) as an intermediate product. The 3-hydroxybutanol is then hydrogenated to form 1,3 butylene glycol.

Typical specifications for acetic acid concentrations in acetaldehyde available for industrial use range from 0.05 wt. % to 0.1 wt. %, based upon the total weight of the acetaldehyde product. It has been unexpectedly discovered that yield efficiencies for production of 1,3 butylene glycol may be increased by aldol condensation of acetaldehyde having a carboxylic acid content of less than 0.04 wt. % based upon the weight of the acetaldehyde.

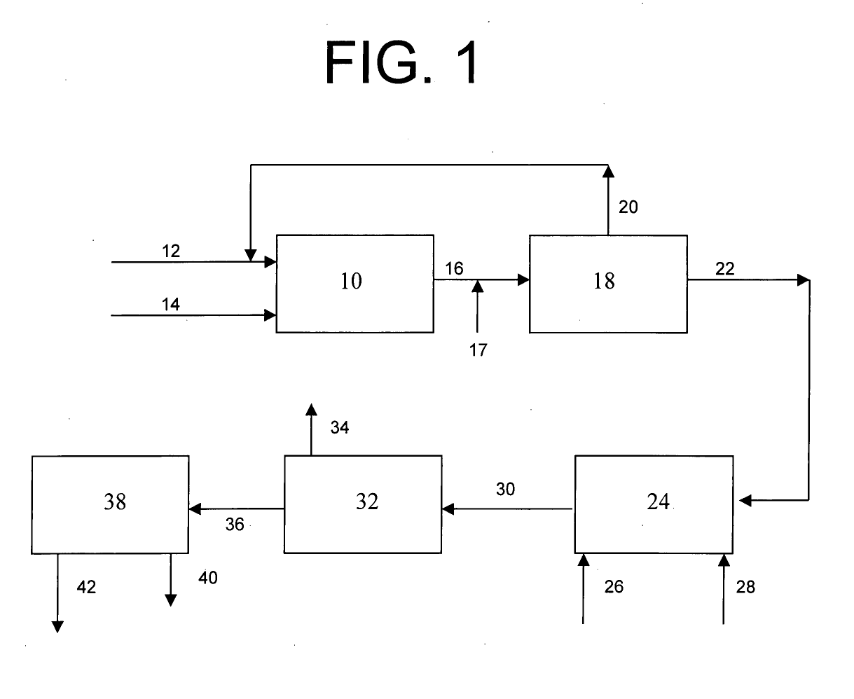

FIG. 1 provides a schematic diagram of an exemplary process for preparation of 1,3 butylene glycol as described herein. Referring to FIG. 1, a continuous operation mode for the production of 1,3 butylene glycol in accordance with an embodiment of this disclosure is illustrated. An aldol condensation of acetaldehyde takes place in a reactor 10 in the presence of a low concentration of an alkali agent such as sodium hydroxide, potassium hydroxide, sodium bicarbonate, or mixtures thereof, acting as a catalyst, to produce the 3-hydroxybutanal. The acetaldehyde and alkali agent are fed simultaneously with the use of metering pumps into reactor 10 to maintain the desired concentration mixtures in accordance the process embodiments described above. The acetaldehyde is added through inlet 12 and the alkali agent is added though inlet 14. In one embodiment, the acetaldehyde and alkali agent are metered into the reactor 10 at a temperature from about 15° C. to about 50° C. and a pressure from about 400 kPa to about 500 kPa. In another embodiment, the acetaldehyde and alkali agent are metered into the reactor 10 at a temperature from about 20° C. to about 50° C. and a pressure from about 300 kPa to about 500 kPa. In still another embodiment, the acetaldehyde and alkali agent are metered into the reactor 10 at a temperature from about 30° C. to about 35° C. and a pressure from about 400 kPa to about 500 kPa. The reaction mixture typically also includes traces of water, crotonaldehyde, and paraldehyde.

In one embodiment, the reaction mixture in reactor 10 should be maintained at a temperature from 20° to about 30° and a pressure from about 306 kPa to about 310 kPa. In another embodiment, the reaction mixture should be maintained at a temperature from 25° to about 26° and a pressure from about 306 kPa to about 308 kPa. In still another embodiment, the reaction mixture should be maintained at a temperature from 25.7° to about 25.8° and a pressure from about 307 kPa to about 310 kPa.

In one embodiment, the alkali agent is present at concentrations from about 2 ppm to about 10 ppm of the total reaction mixture. In another embodiment, the alkali agent is present at concentrations from about 3 ppm to about 5 ppm of the total reaction mixture. In still embodiment, the alkali agent is present at concentrations from about 3 ppm to about 5 ppm of the total reaction mixture.

The aldol condensation reaction is allowed to proceed while stirring the contents of the reactor. In one embodiment, the average acetaldehyde residence time in reactor 10 is about 60 minutes to about 180 minutes. In another embodiment, the average acetaldehyde residence time is about 90 minutes to about 150 minutes. In still another embodiment, the average residence time for acetaldehyde in reactor 10 is about 96 minutes to about 131 minutes.

As the aldol condensation reaction proceeds in reactor 10, a crude product stream 16 is continuously withdrawn from reactor 10. The crude product stream 16 contains unreacted acetaldehyde, trimers of acetaldehyde, alkali agent, and 3-hydroxybutanal.

The crude product stream 16 is treated with an acid such as acetic acid to deactivate the alkali agent catalyst and routed to stripper distillation column 18 having a top portion and a bottom portion for lights ends stripping. Specifically, unreacted acetaldehyde is removed in the overhead 20 from stripper 18 and recycled to reactor 10 through acetaldehyde fed 12. In one embodiment, the top portion of stripper 18 is maintained at a temperature of about 50° C. to about 52° C. and a pressure from about 265 kPa to about 270 kPa and the bottom portion of stripper 18 is maintained at a temperature of about 117° C. to about 120° C. and a pressure from about 275 kPa to about 285 kPa. In another embodiment, the top portion of stripper 18 is maintained at a temperature of about 51° C. to about 51.5° C. and a pressure from about 266 kPa to about 267 kPa and the bottom portion of stripper 18 is maintained at a temperature of about 118° C. to about 119° C. In still another embodiment, the top portion of stripper 18 is maintained at a temperature of about 51° C. to about 51.2° C. and a pressure from about 266 kPa to about 267 kPa and the bottom portion of stripper 18 is maintained at a temperature of about 118° C. to about 118.2° C. and a pressure from about 280 kPa to about 281 kPa. The acetaldehyde recycle stream may be purified to remove crotonaldehyde in the stripper 18 prior to recycle to the reactor.

An isolated 3-hydroxybutanal product stream 22 is removed from the bottom portion of stripper 18 and routed to a liquid phase hydrogenation reduction reactor 24. The 3-hydroxybutanal stream 22, a hydrogen stream 26, and an aqueous Raney nickel catalyst solution stream 28 are metered simultaneously into reactor 24. Other catalysts such as palladium, platinum, and ruthenium may be used although these catalysts are traditionally more expensive. The aqueous Raney nickel catalyst stream may contain from about 0.1 wt. % to about 20 wt. % catalyst. In one embodiment, about 962 cubic meters of hydrogen (volume at standard temperature and pressure of 23° C. and one atmosphere) per hour are fed to the reactor 24. In one embodiment, the hydrogenation reactor 24 is maintained at a temperature of 50° C. to about 200° C. and a pressure from about 101 kPa to about 8000 kPa. In another embodiment, the hydrogenation reactor 24 is maintained at a temperature from about 90° C. to about 110° C. and a pressure from about 3000 kPa to about 5000 kPa. In still another embodiment, the hydrogenation reactor 24 is maintained at a temperature from about 100° C. to about 101° C. and a pressure from about 4000 kPa to about 4300 kPa. The average residence time for the components in reactor 24 is from about one minute to about five hours.

A crude reaction product stream 30 containing 1,3 butylene glycol is removed from hydrogenation reactor 24 and routed to distillation column 32 having a top portion and a bottom portion to remove light ends such as ethylene and butanol in a top stream 34 which may be disposed of or used as process fuel. A 1,3 butylene glycol product stream is removed as bottom stream 36. In one embodiment, the top portion of the distillation column 32 is maintained at a temperature of about 80° C. to about 120° C. and a pressure from about 50 kPa to about 150 kPa and the bottom portion of the distillation column 32 is maintained at a temperature of about 120° C. to about 160° C. and a pressure from about 101 kPa to about 200 kPa. In another embodiment, the top portion of the finishing column 32 is maintained at a temperature of about 90° C. to about 100° C. and a pressure from about 90 kPa to about 110 kPa and the bottom portion of the finishing column 32 is maintained at a temperature of about 140° C. to about 142° C.

Product stream 36 is routed to a vacuum distillation-finishing column 38 to remove additional light ends, water, and aldols in stream 40. A finished 1,3 butylene glycol product stream 42 is taken from finishing column 38 as stream 42. In one embodiment, finishing column 38 is maintained at a temperature from about 82 C to about 116° C. and a pressure from about 50 Pa to about 101 kPa.

The improved processes described herein may be used in continuous commercial reactor systems to produce 1,3 butylene glycol at rates of at least 0.35 liter of crude 1,3 butylene glycol product per hour per liter of reaction mixture. Moreover, these processes may be used to achieve these reaction rates in continuous reaction systems in large volume reaction mixtures of commercial reactors.

It is understood the process described herein may be carried in process other than the continuous process described in connection with FIG. 1. For example, the process may be carried out in sequential steps by first producing 3-hydroxybutanal as described herein and then producing the 1,3 butylene glycol using the 3-hydroxybutanal so produced, in a separate process. Additionally, the processes described here in may be practiced by batch-wise production of the 3-hydroybutanal and/or the 1,3 butylene glycol. It is also understood that the processes described herein may be practiced by preparation of 1,3 butylene glycol by hydrogenation of 3-hydroybutanal having the compositional characteristics of a product produced by aldol condensation of acetaldehyde having a carboxylic acid concentration of less than 0.04 wt. %.

Exemplary Data

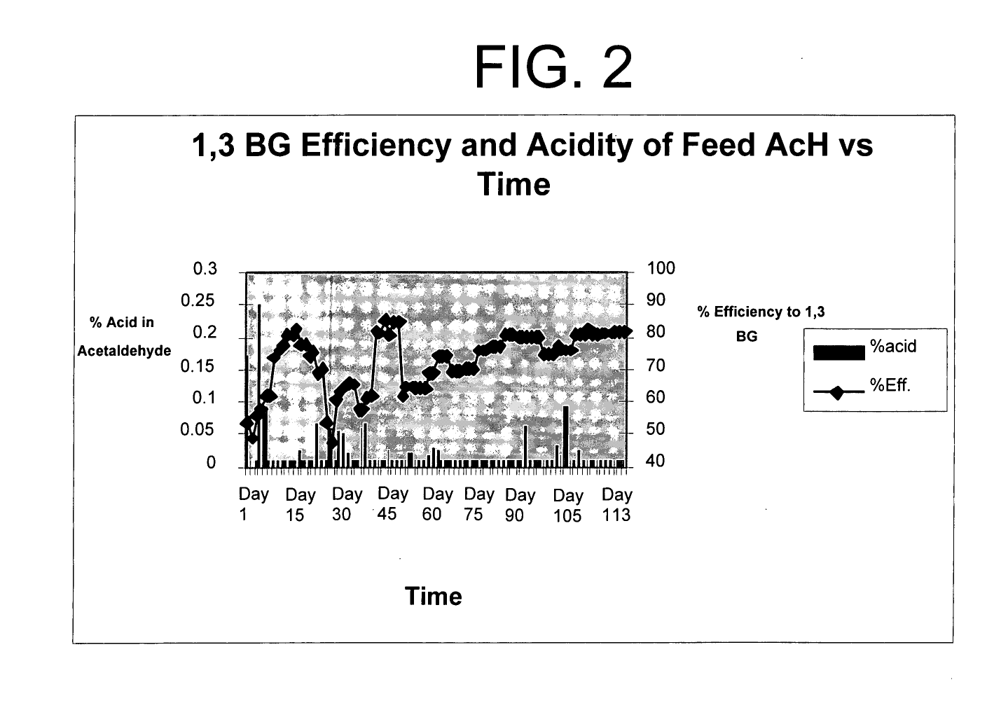

A commercial 1,3 butylene glycol production process in accordance the type described with reference to FIG. 1 was operated for a period of one hundred thirteen (113) days. The process conditions were held constant for the entire period. The average 1,3 butylene glycol production efficiency yield for each day during this period is plotted on the graph of FIG. 2. Also plotted on the graph of FIG. 2 is the average acid concentration of the acetaldehyde feed to the reactor for each day.

As seen from examining the data represented in FIG. 2, there is a direct correlation between the acid concentration in the acetaldehyde feed and the efficiency of the reactor. Specifically, the lower the acid concentration, generally the higher the efficiency yield of the reactor. Additionally, it is seen that production efficiencies of greater than 75% were achieved through the use of acetaldehyde with acid levels of about 0.04% wt. % or less. It is also seen that 1,3 butylene glycol efficiency yields of greater than 80% were attained with very low acid concentrations in the acetaldehyde feed. On two days, high efficiency yields are seen in conjunction with acid concentrations higher than 0.04 wt. %. These are considered to be anomalous data points, the explanation of which is uncertain. However, it is believed that the acid concentrations measured may have been inaccurate on these two days.

All patents and publications referred to herein are hereby incorporated by reference in their entireties.

Although the present invention and its advantages have been described in detail, it should be understood that various changes, substitutions, and alterations could be made without departing from the spirit and scope of the invention as defined by the following claims.

1. A process for the preparation of 1,3-butylene glycol comprising:
(a) aldol condensation of acetaldehyde comprising a carboxylic acid concentration less than or equal to about 0.04 wt. % in the presence of an alkali agent to obtain a reaction mixture comprising 3-hydroxy butanal; and
(b) hydrogenating at least a portion of the 3-hydroxy butanal prepared in step (a) to obtain 1,3-butylene glycol.

2. The process of claim 1 wherein the process is continuous.

3. The process of claim 2 wherein the aldol condensation of the acetaldehyde takes place at a temperature of about 20° C. to about 30° C.

4. The process of claim 1 wherein the alkali agent is present at a concentration of about 2 ppm to about 10 ppm based upon the weight of the acetaldehyde.

5. The process of claim 1 wherein the carboxylic acid concentration of less than or equal to about 0.04 wt. % is the concentration of acetic acid.

6. The process of claim 5 wherein the 1,3 butylene glycol is produced at an efficiency yield of greater than about 75%.

7. The process of claim 4 wherein the alkali agent is selected from the group consisting of sodium hydroxide, potassium hydroxide, sodium bicarbonate, and mixtures thereof.

8. The process of claim 5 wherein the carboxylic acid concentration of the acetaldehyde is less than or equal to about 0.02 wt. %.

9. The process of claim 7 wherein the alkali agent is present at a concentration of about 3 ppm to about 5 ppm based upon the weight of the acetaldehyde.

10. The process of claim 9 wherein the alkali agent is sodium hydroxide present at a concentration of about 3 ppm to about 4 ppm.

11. The process of claim 10 wherein the 1,3 butylene glycol is produce at an efficiency yield of greater than about 80%.

12. The process of claim 8 wherein the carboxylic acid concentration of the acetaldehyde is less than or equal to about 0.01 wt. %.

13. The process of claim 2 wherein 1,3 butylene glycol is produced at rate of at least 0.35 liter of crude 1,3 butylene glycol product per hour per liter of reaction mixture.
